(19) United States
(12) Patent Application Publication
(10) Pub. No.: US 2024/0091747 A1
(43) Pub. Date: Mar. 21, 2024

(54) Ni-BASED CATALYST FOR UNSTEADY-STATE TRACE COX METHANATION REACTION, AND PREPARATION METHOD THEREOF

(71) Applicant: Tsinghua University, Beijing (CN)

(72) Inventors: Binhang YAN, Beijing (CN); Zhihe ZHANG, Beijing (CN)

(21) Appl. No.: 17/933,523

(22) Filed: Sep. 20, 2022

(51) Int. Cl. B01J 23/755; B01J 23/10; B01J 37/00; B01J 37/02; B01J 37/03; B01J 37/04; B01J 37/06; B01J 37/08; C07C 1/12; G01N 30/68

(52) U.S. Cl. CPC B01J 23/755; B01J 23/10; B01J 37/009; B01J 37/0236; B01J 37/035; B01J 37/04; B01J 37/06; B01J 37/08; C07C 1/12; G01N 30/68; C07C 2523/10; C07C 2523/755; G01N 2030/025

(57) ABSTRACT

Disclosed are a Ni-based catalyst for an unsteady-state trace CO, methanation reaction, and a preparation method thereof. The Ni-based catalyst according to the disclosure is composed of an active metal Ni and an oxide support, wherein the oxide support is composed of a rare earth oxide (REO) and an inert oxide; and the rare earth oxide and the inert oxide exhibit an excellent synergistic effect to ensure both high reactivity and weak adsorption and desorption interference. The Ni-based catalyst has a mass percentage of the active metal Ni of 40 wt. % to 90 wt. %, and a mass percentage of the REO of 0.5 wt. % to 20 wt. %, a balance being the inert oxide. The preparation method of the disclosure is a co-precipitation method, including a direct mixing for co-precipitation, a constant-speed dropwise addition for co-precipitation, and a microchannel mixing for co-precipitation.

### TECHNICAL FIELD

The present disclosure relates to the technical fields of catalytic materials preparation and instrument analysis, and in particular to a Ni-based catalyst for an unsteady-state trace CO, methanation reaction, and a preparation method thereof.

### BACKGROUND

In many scenarios of scientific research and industrial production, there is a demand for the detection of a content of trace CO or CO₂. The content of CO, can be as low as a few or tens of parts per million (ppm). Common detection method is Gas chromatography (GC), in which a suitable chromatographic column could be used to separate CO and CO₂, the chromatographic column is connected to a methanation furnace at its rear end, and in the methanation furnace, CO and CO₂ each react with H₂ to generate CH₄, and then a content of each of CO and CO₂ could be determined through a sensitive response of chromatography flame ionization detection (FID) to trace CH₄. Currently, the methanation furnace is generally set at a working temperature of 400° C. or higher. This is because, on the one hand, the existing commercial Ni-based catalysts can only show high methanation activity at about 400° C., and on the other hand, the higher temperature results in great reduction in quantity of physical adsorption on the catalyst surface, making a chemical adsorption reaction reach equilibrium as soon as possible. However, for the chromatographic upgrading in the future, the methanation furnace will be improved to lower the working temperature and reduce the energy consumption. Therefore, it is required to develop a Ni-based methanation catalyst with high activity at a low temperature.

Reactions of methanation catalysts reported in literatures are evaluated as steady-state reactions, and a ratio of H₂ to CON in a reaction gas is usually a stoichiometric ratio. Given that the reaction in the methanation furnaces is performed under a condition of unsteady-state intermittent pulse and the ratio of H₂ to COₓ in a reaction gas is usually far beyond the stoichiometric ratio, it is further required to develop a Ni-based catalyst with high activity at a low temperature which is suitable for the methanation reaction of an unsteady-state trace CON.

A common Ni-based catalyst is usually obtained by loading an active metal Ni onto a metal oxide support, and the support mainly plays a role of supporting and fixing the active metal Ni. It is generally believed that, in a CO₂ methanation reaction, the dissociation of H₂ occurs on Ni particles, the support is responsible for adsorbing and activating CO₂, and a synergistic effect of the active metal Ni and the support promotes the generation of CH₄. Studies have shown that, compared with the use of an inert oxide such as SiO₂, MgO, and Al₂O₃ as a support for a Ni-based catalyst, the use of a rare earth oxide (REO) such as Eu₂O₃, Sm₂O₃, La₂O₃, and CeO₂ as the support could significantly improve the activity of CO₂ methanation. This is because homogeneously-dispersed Ni could partly form interfacial active sites with nearby metal oxides, and carbon species (derived from CO₂) adsorbed at the sites could be quickly hydrogenated to produce CH₄. When the co-precipitation is adopted for the preparation of the catalyst, the mutual dispersion of Ni and a metal oxide could be strengthened, resulting in smaller size of Ni particles, which makes it possible to increase the number of active sites at a metal-support interface and thereby lead to superior low-temperature methanation activity.

The steady-state reaction experiment is generally used for evaluating a reaction. In such experiment, the species evolution on a surface of the catalyst is in a steady state, and the adsorption and desorption of carbon species on the metal oxide support reach a dynamic equilibrium. However, in an actual operation of a methanation furnace, during a unit sample injection time, a trace reaction gas passes through a catalyst bed in a very short period of time, and the catalyst bed is purged with H₂ in the remaining time. During the very short period of time in which the reaction gas passes through the catalyst bed, if CO₂ adsorbed on the metal oxide support cannot be completely hydrogenated, CH₄ will be continuously generated during the subsequent H₂ purging process. According to the chromatographic detection principle, such CH₄ slowly generated cannot be completely detected in the unit sample injection time, which is reflected by the ultra-long tailing of a CH₄ peak in a chromatogram, and a calculated COX conversion rate will be greatly reduced, which greatly reduces the detection accuracy.

Preliminary experimental results show that a Ni-based catalyst with an REO as a support which works well in a steady state works poorly in an unsteady-state pulse reaction; this is because the REO support has a strong adsorption effect for CO₂. It is reported by Tatsuro Horiuchi et al. that the addition of different REOs into Al₂O₃ makes it possible to significantly increase a retention time of CO₂, and a value of adsorption heat would be increased sharply with the increase of the addition amount (see Effect of added basic metal oxides on CO₂ adsorption on alumina at elevated temperatures, Applied Catalysis A: General 167.2 (1998): 195-202). It is reported by Thien An Le et al. that the high activity of a Ni/CeO₂ catalyst in CO and CO₂ methanation reactions is inseparable from the strong adsorption of the catalyst for CO₂ (see CO and CO₂ methanation over supported Ni catalysts, Catalysis Today 293 (2017): 89-96). For Ni-based catalysts with the REO as a support, since Ni on the interface of the catalyst could provide a large amount of H, CO₂ adsorbed by the REO at the interface could be rapidly hydrogenated to generate CH₄. While the REO, which is far away from the interface and accounts for the vast majority, mainly plays a supporting role, and CO₂ adsorbed thereon could only be hydrogenated slowly to generate CH₄. The chromatographic detection results show that this part of CO₂ is not converted. That is, the catalyst shows poor unsteady-state activity. In addition, because a CO, content to be detected is on the order of ppm, no matter how small an amount of CO, adsorbed on the catalyst is, its proportion in a total amount cannot be ignored. In conclusion, the Ni-based catalysts with the REO as a support are not suitable for the application scenarios proposed herein.

### SUMMARY

The present disclosure is intended to provide a Ni-based catalyst and a preparation method thereof. The Ni-based catalyst is suitable for an unsteady-state pulse trace COX methanation reaction, and makes it possible to achieve 98% or more methanation conversion at 200° C. to 300° C. The chromatographic results show that the peak of the product CH₄ does not have a broadening or tailing phenomenon. The Ni-based catalyst of the present disclosure makes it possible to replace the existing catalyst in a methanation furnace arranged for chromatography.

The Ni-based catalyst provided in the present disclosure is composed of an active metal Ni and an oxide support, wherein the oxide support is composed of an REO and an inert oxide; and the rare earth oxide and the inert oxide exhibit an excellent synergistic effect to ensure both high reactivity and weak adsorption and desorption interference. The Ni-based catalyst has a mass percentage of the active metal Ni of 40 wt. % to 90 wt. %, and a mass percentage of the REO of 0.5 wt. % to 20 wt. %, a balance being the inert oxide.

The preparation method provided in the present disclosure is a co-precipitation method, including a direct mixing for co-precipitation, a constant-speed dropwise addition for co-precipitation, and a microchannel mixing for co-precipitation. A precipitate obtained by the co-precipitation is separated through a centrifugation, a drying, and a calcination to obtain the Ni-based catalyst with high activity at a low temperature.

The method for preparing the Ni-based catalyst comprises steps of:
(1) adding 0.005 mol to 0.10 mol of a soluble salt of the active metal Ni, 0.0002 mol to 0.10 mol of a soluble salt of a metal of the REO, and 0.005 mol to 0.80 mol of a soluble salt of a metal of the inert oxide to 50 mL to 500 mL of a solvent to form a mixture I, and stirring the mixture I at 25° C. for dissolution to obtain a mixed metal solution;
(2) adding 0.02 mol to 1.50 mol of a solid precipitating agent to 50 mL to 500 mL of a solvent to form a mixture II, and stirring the mixture II at 25° C. for dissolution to obtain a precipitating agent solution;
(3) subjecting the mixed metal solution obtained in step (1) and the precipitating agent solution obtained in step (2) to a direct mixing, a dropwise mixing, or a microchannel mixing for co-precipitation to obtain a co-precipitated slurry;
(4) thoroughly stirring the co-precipitated slurry, centrifuging the co-precipitated slurry at 2,000 r/min to 5,000 r/min for 3 min to 5 min to collect a precipitate, and washing the precipitate by ultrasonically dispersing the precipitate in a washing agent; and repeating the centrifuging and washing operations for 1 to 4 times to obtain a washed precipitate; and
(5) drying the washed precipitate at 80° C. to 120° C. for 8 h to 20 h, grinding a blocky solid obtained after the drying into a homogeneous powder, and subjecting the homogeneous powder to a calcination in a muffle furnace at 300° C. to 500° C. for 1 h to 5 h to obtain the Ni-based catalyst.

Provided is also use of the Ni-based catalyst in an unsteady-state pulse CO, methanation reaction, and specifically, the use is performed as follows:
packing the Ni-based catalyst for an unsteady-state pulse COₓ methanation reaction into a methanation furnace, and reducing the Ni-based catalyst at 300° C. to 450° C. for 1 h to 2 h to allow a pretreatment; during a single chromatographic acquisition time, intermittently pumping a reaction gas in a chromatographic quantification loop through a shut-off valve into the methanation furnace at a flow rate of 5 mL/min to 30 mL/min, and continuously introducing a carrier gas into the methanation furnace at a flow rate of 20 mL/min to 60 mL/min; after trace COₓ in the reaction gas is converted into CH₄ through a methanation reaction, determining a concentration of CH₄ by a flame ionization detector (FID) to obtain a content of the trace COₓ in the reaction gas, where
a content of the trace COₓ in the reaction gas is in the range of 2 ppm to 5,000 ppm;
the methanation reaction is performed at a temperature of 200° C. to 300° C. and a pressure of 1 bar;
a volume of the chromatographic quantification loop is 20 μL to 1,000 μL; and
the carrier gas is a high-purity H₂ or a mixture of H₂ and Ar, and the mixture of H₂ and Ar includes 10% to 80% of H₂.

The Ni-based catalyst provided by the present disclosure has the following advantages:
1. In the Ni-based catalyst of the present disclosure, Ni and the support metal are co-precipitated, such that the active metal Ni and the support oxide could be thoroughly mixed. Since the particle size of Ni is small, an interaction between Ni and the support is strong.
2. The support in the Ni-based catalyst of the present disclosure is composed of a reducible support and an inert support, which combines the advantages of the two and ensures prominent reactivity and weak adsorption and desorption interference. Therefore, the Ni-based catalyst is suitable for an unsteady-state pulse COₓ methanation reaction.
3. The Ni-based catalyst of the present disclosure makes it possible to achieve 98% or more methanation conversion at a low temperature of 200° C. to 300° C. when used for an unsteady-state pulse COₓ methanation reaction. The chromatographic results show that the peak of the product CH₄ does not have broadening or tailing phenomenon. The Ni-based catalyst of the present disclosure has the potential to replace the existing catalyst in a methanation furnace arranged for chromatography.
4. The preparation method of the Ni-based catalyst of the present disclosure is a co-precipitation method, and makes it possible to realize the batch preparation of the catalyst, with simple and safe operations and high repeatability.

In a Ni-based catalyst according to the present disclosure, an inert oxide is used as a primary support, which plays a major supporting and fixing role and meanwhile shows weak adsorption for CO₂, without causing interference, and on the other hand, the REO is used as a secondary support, which could reduce the content of REO but ensure the dispersion of REO, resulting in that a large number of Ni-REO support interfaces are still formed to ensure the CO₂ methanation activity. The catalyst combines the properties of the two oxides and is fully suitable for the unsteady-state pulse COₓ methanation reaction proposed herein.

### DETAILED DESCRIPTION OF THE EMBODIMENTS

The Ni-based catalyst provided in the present disclosure is composed of an active metal Ni and an oxide support, wherein the oxide support is composed of an REO and an inert oxide; and the rare earth oxide and the inert oxide exhibit an excellent synergistic effect to ensure both high reactivity and weak adsorption and desorption interference. The Ni-based catalyst has a mass percentage of the active metal Ni of 40 wt. % to 90 wt. %, and a mass percentage of the REO of 0.5 wt. % to 20 wt. %, a balance being the inert oxide.

In the present disclosure, the REO in the oxide support of the Ni-based catalyst may be one or more selected from the group consisting of CeO₂, La₂O₃, Eu₂₀₃, and Sm₂₀₃, and the REO accounts for 0.5 wt. % to 20 wt. % of a total mass of the catalyst. The inert oxide in the oxide support may be one or more selected from the group consisting of SiO₂, MgO, and Al₂O₃, and the inert oxide accounts for 2 wt. % to 60 wt. % of a total mass of the catalyst.

The preparation method provided in the present disclosure is a co-precipitation method, including a direct mixing for co-precipitation, a constant-speed dropwise addition for co-precipitation, and a microchannel mixing for co-precipitation. A precipitate obtained by the preparation method is separated through a centrifugation, a drying, and a calcination to obtain the Ni-based catalyst with high activity at a low temperature. The method for preparing the Ni-based catalyst comprises steps of:
(1) adding 0.005 mol to 0.10 mol of a soluble salt of the active metal Ni, 0.0002 mol to 0.10 mol of a soluble salt of a metal of the REO, and 0.005 mol to 0.80 mol of a soluble salt of a metal of the inert oxide to 50 mL to 500 mL of a solvent to form a mixture I, and stirring the mixture I at 25° C. for dissolution to obtain a mixed metal solution;
(2) adding 0.02 mol to 1.50 mol of a solid precipitating agent to 50 mL to 500 mL of a solvent to form a mixture II, and stirring the mixture II at 25° C. for dissolution to obtain a precipitating agent solution;
(3) subjecting the mixed metal solution obtained in step (1) and the precipitating agent solution obtained in step (2) to a direct mixing, a dropwise mixing, or a microchannel mixing for co-precipitation to obtain a co-precipitated slurry;
(4) thoroughly stirring the co-precipitated slurry, centrifuging the co-precipitated slurry at 2,000 r/min to 5,000 r/min for 3 min to 5 min to collect a precipitate, and washing the precipitate by ultrasonically dispersing the precipitate in a washing agent; and repeating the centrifuging and washing operations for 1 to 4 times; and
(5) drying the washed precipitate at 80° C. to 120° C. for 8 h to 20 h, grinding a blocky solid obtained after the drying into a powder, and subjecting the powder to a calcination in a muffle furnace at 300° C. to 500° C. for 1 h to 5 h to obtain the Ni-based catalyst.

The present disclosure is further described below with reference to examples, but the embodiments of the present disclosure are not limited thereto.

### Example 1

(1) 0.01 mol of nickel nitrate hexahydrate, 0.000426 mol of cerium nitrate hexahydrate, and 0.0079 mol of aluminum nitrate nonahydrate were added to 100 mL of absolute ethanol, and a resulting mixture was stirred at 25° C. for dissolution to obtain a mixed metal solution.
(2) 0.06 mol of oxalic acid was added to 100 mL of absolute ethanol, and a resulting mixture was stirred at 25° C. for dissolution to obtain a precipitating agent solution.
(3) The mixed metal solution and the precipitating agent solution were directly mixed for co-precipitation to obtain a co-precipitated slurry.
(4) The co-precipitated slurry was thoroughly stirred and then centrifuged at 4,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 2 times repeatedly.
(5) The washed precipitate obtained in above (4) was dried at 100° C. for 15 h, ground into a homogeneous powder, and subjected to a calcination in a muffle furnace at 400° C. for 4 h to obtain a Ni-based catalyst in the form of powder.

The catalyst in the example was subjected to a granulation, and 100 mg of the catalyst with 40 to 60 mesh was taken and packed in a quartz tube. Before an activity test, the catalyst was activated through reduction at 400° C. for 2 h with 40 mL/min pure H₂ as a reducing gas.

After the catalyst was reduced, the activity test was directly conducted in situ as follows: during a single chromatographic acquisition time of 9.8 min, a reaction gas consisting of 100 ppm of CO 2 and 100 ppm of CH 4 (internal standard) in a chromatographic quantification loop (with a volume of 250 μL) was intermittently pumped through a shut-off valve into a methanation furnace at a flow rate of 20 mL/min, and a carrier gas (i.e. a high-purity H₂) was continuously introduced into the methanation furnace at a flow rate of 40 mL/min. After trace CO₂ in the reaction gas was converted into CH₄ through a methanation reaction, a concentration of the product CH₄ was determined by an FID (peak area: 363,497) and a concentration of an internal standard CH₄ was determined by an FID (peak area: 368,454), and the two were compared to calculate a conversion rate of CO₂ methanation corresponding to the catalyst, which was 98.65%. The methanation reaction was performed at a temperature of 270° C. and a pressure of 1 bar. The Shimadzu Nexis GC-2030 chromatography system was used for detection. It can be known that the catalyst is suitable for an unsteady-state COₓ methanation reaction and exhibits excellent reactivity.

### Example 2

(1) 0.004 mol of nickel nitrate hexahydrate, 0.00027 mol of samarium nitrate hexahydrate, and 0.01094 mol of tetraethyl silicate were added to 50 mL of absolute ethanol, and a resulting mixture was stirred at 25° C. for dissolution to obtain a mixed metal solution.
(2) 10 mL of ammonia water was added to 40 mL of absolute ethanol, and a resulting mixture was thoroughly mixed at 25° C. to obtain a precipitating agent solution.
(3) The mixed metal solution and the precipitating agent solution were mixed through constant-speed dropwise addition for co-precipitation to obtain a co-precipitated slurry.
(4) The co-precipitated slurry was thoroughly stirred and then centrifuged at 4,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 4 times repeatedly.
(5) The washed precipitate obtained in above (4) was dried at 110° C. for 15 h, ground into a homogeneous powder, and subjected to a calcination in a muffle furnace at 400° C. for 3 h to obtain a Ni-based catalyst in the form of powder.

The catalyst in the example was subjected to a granulation, and 100 mg of the catalyst with 40 to 60 mesh was taken and packed in a quartz tube. Before an activity test, the catalyst was activated through reduction at 400° C. for 2 h with 40 mL/min pure H₂ as a reducing gas.

After the catalyst was reduced, the activity test was directly conducted in situ as follows: during a single chromatographic acquisition time of 9.8 min, a reaction gas consisting of 1,000 ppm of CO₂ and 1,000 ppm of CH₄ (internal standard) in a chromatographic quantification loop (with a volume of 250 μL) was intermittently pumped through a shut-off valve into a methanation furnace at a flow rate of 20 mL/min, and a carrier gas (i.e. a high-purity H₂) was continuously introduced into the methanation furnace at a flow rate of 40 mL/min. After trace CO₂ in the reaction gas was converted into CH₄ through a methanation reaction, a concentration of the product CH₄ was determined by an FID (peak area: 4,079,000) and a concentration of an internal standard CH₄ was determined by an FID (peak area: 4,146,243), and the two were compared to calculate a conversion rate of CO₂ methanation corresponding to the catalyst, which was 98.38%. The methanation reaction was performed at a temperature of 300° C. and a pressure of 1 bar. The Shimadzu Nexis GC-2030 chromatography system was used for detection. It can be known that the catalyst is suitable for an unsteady-state COₓ methanation reaction and exhibits excellent reactivity.

### Example 3

(1) 0.04 mol of nickel nitrate hexahydrate, 0.00175 mol of europium nitrate hexahydrate, 0.00175 mol of cerium nitrate hexahydrate, and 0.02878 mol of aluminum nitrate nonahydrate were added to 100 mL of absolute ethanol, and a resulting mixture was stirred at 25° C. for dissolution to obtain a mixed metal solution.
(2) 0.2 mol of oxalic acid was added to 100 mL of absolute ethanol, and a resulting mixture was stirred at 25° C. for dissolution to obtain a precipitating agent solution.
(3) The mixed metal solution and the precipitating agent solution were mixed through a microchannel for co-precipitation to obtain a co-precipitated slurry.
(4) The co-precipitated slurry was thoroughly stirred and then centrifuged at 4,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 3 times repeatedly.
(5) The washed precipitate obtained in above (4) was dried at 105° C. for 14 h, ground into a homogeneous powder, and subjected to a calcination in a muffle furnace at 400° C. for 2 h to obtain a Ni-based catalyst in the form of powder.

The catalyst in the example was subjected to a granulation, and 100 mg of the catalyst with 40 to 50 mesh was taken and packed in a quartz tube. Before an activity test, the catalyst was activated through reduction at 400° C. for 2 h with 40 mL/min pure H₂ as a reducing gas.

After the catalyst was reduced, the activity test was directly conducted in situ as follows: during a single chromatographic acquisition time of 9.8 min, a reaction gas consisting of 100 ppm of CO 2 and 100 ppm of CH 4 (internal standard) in a chromatographic quantification loop (with a volume of 250 μL) was intermittently pumped through a shut-off valve into a methanation furnace at a flow rate of 20 mL/min, and a carrier gas (i.e. a high-purity H₂) was continuously introduced into the methanation furnace at a flow rate of 40 mL/min. After trace CO₂ in the reaction gas was converted into CH₄ through a methanation reaction, a concentration of the product CH₄ was determined by an FID (peak area: 361774) and a concentration of an internal standard CH₄ was determined by an FID (peak area: 368552), and the two were compared to calculate a conversion rate of CO₂ methanation corresponding to the catalyst, which was 98.16%. The methanation reaction was performed at a temperature of 290° C. and a pressure of 1 bar. The Shimadzu Nexis GC-2030 chromatography system was used for detection. It can be known that the catalyst is suitable for an unsteady-state COₓ methanation reaction and exhibits excellent reactivity.

### Example 4

(1) 0.04 mol of nickel nitrate hexahydrate, 0.04948 mol of zirconium nitrate pentahydrate, and 0.005497 mol of cerium nitrate hexahydrate were added to 200 mL of absolute ethanol, and a resulting mixture was stirred at 25° C. for dissolution to obtain a mixed metal solution.
(2) 0.3 mol of oxalic acid was added to 200 mL of absolute ethanol, and a resulting mixture was stirred at 25° C. for dissolution to obtain a precipitating agent solution.
(3) The mixed metal solution and the precipitating agent solution were mixed through a microchannel for co-precipitation to obtain a co-precipitated slurry.
(4) The co-precipitated slurry was thoroughly stirred and then centrifuged at 4,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 3 times repeatedly.
(5) The washed precipitate obtained in above (4) was dried at 100° C. for 12 h, ground into a homogeneous powder, and subjected to a calcination in a muffle furnace at 400° C. for 2 h to obtain a Ni-based catalyst in the form of powder.

The catalyst in the example was subjected to a granulation, and 100 mg of the catalyst with 40 to 50 mesh was taken and packed in a quartz tube. Before an activity test, the catalyst was activated through reduction at 400° C. for 2 h with 40 mL/min pure H₂ as a reducing gas.

After the catalyst was reduced, the activity test was directly conducted in situ as follows: during a single chromatographic acquisition time of 9.8 min, a reaction gas consisting of 5,000 ppm of CO 2 and 5,000 ppm of CH 4 (internal standard) in a chromatographic quantification loop (with a volume of 250 μL) was intermittently pumped through a shut-off valve into a methanation furnace at a flow rate of 20 mL/min, and a carrier gas (i.e. a high-purity H₂) was continuously introduced into the methanation furnace at a flow rate of 40 mL/min. After trace CO₂ in the reaction gas was converted into CH₄ through a methanation reaction, a concentration of the product CH₄ was determined by an FID (peak area: 17,993,400) and a concentration of an internal standard CH₄ was determined by an FID (peak area: 18,351,600), and the two were compared to calculate a conversion rate of CO₂ methanation corresponding to the catalyst, which was 98.05%. The methanation reaction was performed at a temperature of 290° C. and a pressure of 1 bar. The Shimadzu Nexis GC-2030 chromatography system was used for detection. It can be known that the catalyst is suitable for an unsteady-state COₓ methanation reaction and exhibits excellent reactivity.

### Example 5

(1) 0.02 mol of nickel nitrate hexahydrate, 0.006716 mol of aluminum nitrate nonahydrate, and 0.0005683 mol of cerium nitrate hexahydrate were added to 100 mL of deionized water, and a resulting mixture was stirred at 25° C. for dissolution to obtain a mixed metal solution.
(2) 0.15 mol of ammonium carbonate was added to 100 mL of deionized water, and a resulting mixture was stirred at 25° C. for dissolution to obtain a precipitating agent solution.
(3) The mixed metal solution and the precipitating agent solution were directly mixed for co-precipitation to obtain a co-precipitated slurry.
(4) The co-precipitated slurry was thoroughly stirred and then centrifuged at 5,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 4 times repeatedly.
(5) The washed precipitate obtained in above (4) was dried at 100° C. for 15 h, ground into a homogeneous powder, and subjected to a calcination in a muffle furnace at 400° C. for 3 h to obtain a Ni-based catalyst in the form of powder.

The catalyst in the example was subjected to a granulation, and 100 mg of the catalyst with 60 to 80 mesh was taken and packed in a quartz tube. Before an activity test, the catalyst was activated through reduction at 400° C. for 1.5 h with 40 mL/min pure H₂ as a reducing gas.

After the catalyst was reduced, the activity test was directly conducted in situ as follows: during a single chromatographic acquisition time of 9.8 min, a reaction gas consisting of 100 ppm of CO and 100 ppm of CH 4 (internal standard) in a chromatographic quantification loop (with a volume of 250 μL) was intermittently pumped through a shut-off valve into a methanation furnace at a flow rate of 20 mL/min, and a carrier gas (i.e. a mixture of H₂ and Ar with 50% of H₂) was continuously introduced into the methanation furnace at a flow rate of 40 mL/min. After trace CO in the reaction gas was converted into CH₄ through the methanation reaction, a concentration of the product CH₄ was determined by an FID (peak area: 355,972) and a concentration of an internal standard CH₄ was determined by an FID (peak area: 359,106), and the two were compared to calculate a conversion rate of CO methanation corresponding to the catalyst, which was 99.13%. The methanation reaction was performed at a temperature of 275° C. and a pressure of 1 bar. The Shimadzu Nexis GC-2030 chromatography system was used for detection. It can be known that the catalyst is suitable for an unsteady-state COₓ methanation reaction and exhibits excellent reactivity.

### Example 6

(1) 0.02 mol of nickel nitrate hexahydrate, 0.008222 mol of aluminum nitrate nonahydrate, and 0.0006437 mol of lanthanum nitrate hexahydrate were added to 100 mL of deionized water, and a resulting mixture was stirred at 25° C. for dissolution to obtain a mixed metal solution.
(2) 0.15 mol of ammonium carbonate was added to 100 mL of deionized water, and a resulting mixture was stirred at 25° C. for dissolution to obtain a precipitating agent solution.
(3) The mixed metal solution and the precipitating agent solution were mixed through a microchannel for co-precipitation to obtain a co-precipitated slurry.
(4) The co-precipitated slurry was thoroughly stirred and then centrifuged at 5,000 r/min for 3 min, and a resulting supernatant was discarded and a precipitate was collected. The precipitate was washed by ultrasonically dispersing in absolute ethanol to obtain a washed precipitate. The resulting co-precipitated slurry was centrifuged and washed for 3 times repeatedly.
(5) The washed precipitate obtained in above (4) was dried at 110° C. for 15 h, ground into a homogeneous powder, and subjected to a calcination in a muffle furnace at 400° C. for 3 h to obtain a Ni-based catalyst in the form of powder.

The catalyst in the example was subjected to a granulation, and 100 mg of the catalyst with 40 to 60 mesh was taken and packed in a quartz tube. Before an activity test, the catalyst was activated through reduction at 400° C. for 2.5 h with 40 mL/min pure H₂ as a reducing gas.

After the catalyst was reduced, the activity test was directly conducted in situ as follows: during a single chromatographic acquisition time of 9.8 min, a reaction gas consisting of 100 ppm of CO, 100 ppm of CO₂, and 100 ppm of CH₄ (internal standard) in a chromatographic quantification loop (with a volume of 250 μL) was intermittently pumped through a shut-off valve into a methanation furnace at a flow rate of 20 mL/min, and a carrier gas (i.e. a mixture of H₂ and Ar with 20% of H₂) was continuously introduced into the methanation furnace at a flow rate of 40 mL/min. After trace CO in the reaction gas was converted into CH₄ through the methanation reaction, a concentration of the product CH₄ of CO methanation was determined by an FID (peak area: 356,873) and a concentration of the product CH₄ of CO₂ methanation was determined by an FID (peak area: 360,069), which were each compared with a concentration of an internal standard CH₄ determined by an FID (peak area: 361,674) to calculate a conversion rate of CO methanation (98.67%) and a conversion rate of CO₂ methanation (99.56%) corresponding to the catalyst. The methanation reaction was performed at a temperature of 300° C. and a pressure of 1 bar. The Shimadzu Nexis GC-2030 chromatography system was used for detection. It can be known that the catalyst is suitable for an unsteady-state COₓ methanation reaction and exhibits excellent reactivity.

1. A Ni-based catalyst for an unsteady-state trace COₓ methanation reaction, the Ni-based catalyst being composed of an active metal Ni and an oxide support, wherein;
the oxide support is composed of a rare earth oxide (REO) and an inert oxide; and
the rare earth oxide and the inert oxide exhibit an excellent synergistic effect to ensure both high reactivity and weak adsorption and desorption interference of COₓ.

2. The Ni-based catalyst of claim 1, wherein the Ni-based catalyst has a mass percentage of the active metal Ni of 40 wt. % to 90 wt. %, and the active metal Ni and the oxide support are homogeneously dispersed and have a strong interaction.

3. The Ni-based catalyst of claim 1, wherein the REO in the oxide support is one or more selected from the group consisting of CeO₂, La₂O₃, Eu₂O₃, and Sm₂O₃, and the REO accounts for 0.5 wt. % to 20 wt. % of a total mass of the catalyst.

4. The Ni-based catalyst of claim 1, wherein the inert oxide in the oxide support is one or more selected from the group consisting of SiO₂, MgO, and Al₂O₃, and the inert oxide accounts for 2 wt. % to 60 wt. % of a total mass of the catalyst.

5. A method for preparing the Ni-based catalyst of claim 1, comprising:
(1) adding 0.005 mol to 0.10 mol of a soluble salt of the active metal Ni, 0.0002 mol to 0.10 mol of a soluble salt of a metal of the REO, and 0.005 mol to 0.80 mol of a soluble salt of a metal of the inert oxide to 50 mL to 500 mL of a solvent to form a mixture I, and stirring the mixture I at 25° C. for dissolution to obtain a mixed metal solution;
(2) adding 0.02 mol to 1.50 mol of a solid precipitating agent to 50 mL to 500 mL of a solvent to form a mixture II, and stirring the mixture II at 25° C. for dissolution to obtain a precipitating agent solution;
(3) subjecting the mixed metal solution obtained in step (1) and the precipitating agent solution obtained in step (2) to a direct mixing, a dropwise mixing, or a microchannel mixing for co-precipitation to obtain a co-precipitated slurry;
(4) thoroughly stirring the co-precipitated slurry, centrifuging the co-precipitated slurry at a speed of 2,000 r/min to 5,000 r/min for 3 min to 5 min to collect a precipitate, and washing the precipitate by ultrasonically dispersing the precipitate in a washing agent; and repeating the centrifuging and washing operations for 1 to 4 times to obtain a washed precipitate; and
(5) drying the washed precipitate at a temperature of 80° C. to 120° C. for 8 h to 20 h, grinding a blocky solid obtained after the drying into a homogeneous powder, and subjecting the homogeneous powder to a calcination in a muffle furnace at a temperature of 300° C. to 500° C. for 1 h to 5 h to obtain the Ni-based catalyst of claim 1.

6. The method of claim 5, wherein the soluble salt of the active metal Ni in step (1) is one or more selected from the group consisting of nickel nitrate, nickel acetate, and nickel chloride.

7. The method of claim 5, wherein each of the soluble salt of a metal of the REO and the soluble salt of a metal of the inert oxide in step (1) is one or more selected from the group consisting of a nitrate, an acetate, and an ethyl ester salt.

8. The method of claim 5, wherein the solvent in step (1) is selected from the group consisting of absolute ethanol and deionized water; and the solvent in step (2) is selected from the group consisting of absolute ethanol and deionized water.

9. The method of claim 5, wherein the precipitating agent used in step (2) is selected from the group consisting of oxalic acid, ammonium carbonate, ammonia water, and sodium hydroxide, and has a concentration of 1 to 5 times a total concentration of metal ions in the mixed metal solution; and the precipitating agent has a concentration of 0.2 mol/L to 4.0 mol/L.

10. A method for determining a content of trace COₓ in a reaction gas by a COX methanation reaction using the Ni-based catalyst for an unsteady-state trace COₓ methanation reaction of claim 1, comprising:
packing the Ni-based catalyst for an unsteady-state trace COₓ methanation reaction of claim 1 into a methanation furnace, and reducing the Ni-based catalyst at a temperature of 300° C. to 450° C. for 1 h to 2 h to allow a pretreatment; during a single chromatographic acquisition time, intermittently pumping a reaction gas in a chromatographic quantification loop through a shut-off valve into the methanation furnace at a flow rate of 5 mL/min to 30 mL/min, and continuously introducing a carrier gas into the methanation furnace at a flow rate of 20 mL/min to 60 mL/min; and after trace CO, in the reaction gas is converted into CH₄ through a methanation reaction, determining a concentration of CH₄ by a flame ionization detector (FID) to obtain a content of the trace COₓ in the reaction gas, wherein
the content of the trace COₓ in the reaction gas is in the range of 2 ppm to 5,000 ppm;
the methanation reaction is performed at a temperature of 200° C. to 300° C. and a pressure of 1 bar;
a volume of the chromatographic quantification loop is in the range of 20 μL to 1,000 μL; and
the carrier gas is a high-purity H₂ or a mixture of H₂ and Ar, and the mixture of H₂ and Ar comprises 10% to 80% of H₂.
